(19) United States
(12) Patent Application Publication
(10) Pub. No.: US 2023/0149420 A1
(43) Pub. Date: May 18, 2023

(54) ANTI-INFLAMMATORY IMMUNOENHANCER

(71) Applicant: SCENT SCIENCE INTERNATIONAL INC., Ibaraki (JP)

(72) Inventors: Ko KOBAYAKAWA, Ibaraki (JP); Reiko KOBAYAKAWA, Ibaraki (JP)

(73) Assignee: SCENT SCIENCE INTERNATIONAL INC., Ibaraki (JP)

(21) Appl. No.: 17/906,962

(22) PCT Filed: Mar. 25, 2021
(86) PCT No.: PCT/JP2021/012593

§ 371 Date: Sep. 21, 2022

(30) Foreign Application Priority Data
Mar. 25, 2020 (JP) 2020-054032

(51) Int. Cl. A61K 31/55; A61K 31/426; A61K 31/381; A61K 31/40; A61K 31/428; A61K 31/4406; A61K 31/54; A61K 31/4409; A61K 31/5415; A61K 31/497; A61K 31/47; A61K 31/495; A61K 31/5375; A61K 31/26; A61K 31/44; A61K 31/341; A61K 31/343; A61P 29/00; A61P 37/04

(52) U.S. Cl. CPC A61K 31/55; A61K 31/426; A61K 31/381; A61K 31/40; A61K 31/428; A61K 31/4406; A61K 31/54; A61K 31/4409; A61K 31/5415; A61K 31/497; A61K 31/47; A61K 31/495; A61K 31/5375; A61K 31/26; A61K 31/44; A61K 31/341; A61K 31/343; A61P 29/00; A61P 37/04

(57) ABSTRACT

The present invention provides an anti-inflammatory type immune-enhancing agent that simultaneously induces an anti-inflammatory action and an immune-enhancing action. An anti-inflammatory type immune-enhancing agent containing at least one kind selected from a heterocyclic compound represented by the formula (I)

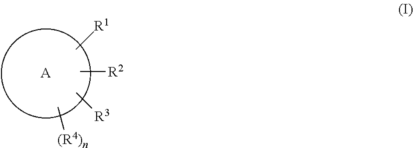

wherein each symbol is as described in the DESCRIPTION, or a salt thereof, and an isothiocyanate compound represented by the formula S═C═N—R⁵ (II) wherein the symbol is as described in the DESCRIPTION, as an active ingredient.

### TECHNICAL FIELD

The present invention relates to a technique for simultaneously inducing an anti-inflammatory action and an immune-enhancing action based on a mechanism of activating latent innate life-protective abilities by sensory stimulation.

### BACKGROUND ART

During the process of evolution, humans and animals have evolved life-protective abilities that respond to various situations. Life-threatened organisms are considered to maximize their survival probability by exerting their latent life-protective abilities. Therefore, if a new technique that induces a potential life-protective action that has been evolved by living organisms can be utilized in the medical field of humans and animals, it is theoretically expected to improve the survival rate and prognosis. However, a biological theoretical basis for developing such a new technique has not been discovered for a long time.

With this background, the present inventors discovered a phenomenon in which a latent life-protective action is induced by transmitting crisis information to the brain through the activation of TRPA1 (transient receptor potential ankyrin 1), which is the sensory receptor, by a group of heterocyclic and straight chain odor molecules that induce innate fear emotions. Furthermore, they developed a technique to obtain therapeutic effects on hypoxic injury and inflammation by using the group of heterocyclic and straight chain odor molecules developed then (these are referred to as thiazoline-related fear odors) (Patent Literature 1).

The immune system is essential for defending living organisms against infection. However, an excessive immune response causes inflammation that also destroys one's own cells and tissues. In order to treat inflammatory diseases, it is necessary to administer anti-inflammatory drugs to suppress excessive immune responses. However, suppression of the immune response means weakening the defense response against infections, sepsis, cancer, and the like. The immune response needs to remain regulated at an appropriate level because both m excessive enhancement and suppression can be detrimental to life and health.

Through activation of glucocorticoid receptors, steroidal anti-inflammatory drugs affect the expression of genes involved in various immune actions and other physiological functions. Thus, steroid drug administration has a superior anti-inflammatory action but simultaneously shows a strong suppressive action on immunity. Therefore, the administration of a steroid drug has the side effect of weakening the ability to attack bacteria infected from the outside world and cancers developed in the body (Non-Patent Literatures 1-3).

Therefore, it would be ideal if there existed a drug that has the contradictory actions of inducing anti-inflammatory action and enhancing immune action; however, such a drug has not been developed.

### CITATION LIST

### Patent Literature

### [PTL 1]

WO2019/177142

### Non Patent Literature

### [NPL 1]

Japanese Journal of Allergology 60(2), 193-198, 2011

### [NPL 2]

Edited and written by Yutaka Mizushima, “Today's Drug Therapy—Commentary and Handbook”, Nankodo

### [NPL 3]

Kazuhiko Yamamoto (ed.), “Handbook for Selecting and Using Steroid Drugs”, YODOSHA CO., LTD.

### [NPL 4]

Miller et al., Nat Immunol 20, 326-336 (2019)

### [NPL 5]

Tikhonova et al., Nature 569, p 222-228 (2019)

### [NPL 6]

Matsuo et al., bioRxiv, doi: https://doi.org/10.1101/2020.05.17.100933 (2020)

### [NPL 7]

Matsuo et al., Commun Biol 4, 101 (2021)

### SUMMARY OF INVENTION

### Technical Problem

The present invention aims to provide a medicament that induces both an anti-inflammatory action and an immune-enhancing action.

### Solution to Problem

The present inventors have proposed a model that thiazoline-related fear odors activate the powerful crisis recognition system in the brain via sensory nerves, and as a result, show an action of maximizing the endogenous life-protecting action acquired in the process of evolution (Non-Patent Literatures 6, 7). They consider that thiazoline-related fear odors may be able to simultaneously induce contradictory actions of anti-inflammation and immune enhancement, which was difficult with the conventional technology, by intervening in the crisis response system of the brain that integrates and controls the state of the entire body.

According to this original hypothesis, they have found that stimulation of thiazoline-related fear odors induces a strong anti-inflammatory action and simultaneously elevates blood levels of monocytes, dendritic cells, neutrophils, and basophils, which have an innate immune action, and lymphocytes, which also have an acquired immune action. Under normal conditions, an increase in these immune cells causes aggravation of the inflammatory state. However, stimulation of thiazoline-related fear odors has made it possible for the first time to induce an anti-inflammatory type immune-enhancing action that simultaneously induces an immune-enhancing action and an anti-inflammatory action, which was difficult to realize with conventional medicaments, by increasing the expression of genes that suppress inflammatory action in innate immune cells and genes that enhance innate immune function.

Therefore, a technique that imparts an anti-inflammatory action and simultaneously potentiates an immunity action, which is difficult with existing anti-inflammatory agents, has been developed using thiazoline-related fear odors by the present invention.

That is, the present invention relates to the following. [1] An anti-inflammatory type immune-enhancing agent comprising, as an active ingredient, at least one kind selected from a heterocyclic compound represented by the formula (I)

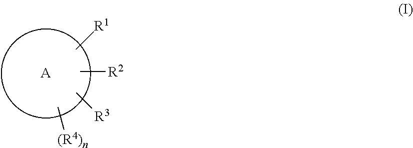

wherein
ring A is a 5- to 7-membered heterocycle containing 1 or 2 hetero atoms selected from a nitrogen atom, an optionally oxidized sulfur atom and an oxygen atom;
R¹, R², R³ and R⁴ are each independently a hydrogen atom, a C₁₋₆ alkyl group, a C₁₋₆ alkoxy group, a halogen atom, an amino group, —SH, a C₁₋₆ alkylthio group, a C₂₋₆ alkenylthio group, a C₁₋₆ alkyl-carbonyl group, a formyl group, a C₆₋₁₀ aryl group, a C₁₋₆ alkoxycarbonyl group, a 5- or 6-membered heteroaryl group, a 5- or 6-membered heteroaryl-C₁₋₆ alkyl group, a 5- or 6-membered heteroaryl-C₁₋₆ alkylthio group, or an oxo group;
R¹ and R² are optionally bonded to each other to form an optionally substituted 5- to 10-membered ring; and
n is 0, 1, or 2, or a salt thereof, and
an isothiocyanate compound represented by the formula (II)

S═C═N—R⁵ (II)

wherein R⁵ is a C₁₋₆ alkyl group, a C₁₋₆ haloalkyl group, a C₂₋₆ alkenyl group, a C₁₋₆ alkylthio-C₁₋₆ alkyl group, a C₆₋₁₀ aryl group, a C₆₋₁₀ aryl-C₁₋₆ alkyl group, or a 5- or 6-membered heteroaryl group.
[2] The agent of [1], wherein the ring A is thiazoline, thiazole, thiazolidine, thiomorpholine, thiophene, pyrrole, morpholine, azepane, pyridine, pyrazine, furan, 2,3-dihydro-4H-1,4-thiazine, imidazole, or tetrahydrofuran.
[3] The agent of [1] or [2], wherein the active ingredient is a heterocyclic compound represented by the formula (I) or a salt thereof.
[4] The agent of [1], wherein the active ingredient is an isothiocyanate compound represented by the formula (II).
[5] Use of at least one kind of compound selected from a heterocyclic compound represented by the formula (I) or a salt thereof, and an isothiocyanate compound represented by the formula (II) for the production of an anti-inflammatory type immune-enhancing agent.
[6] The use of [5], wherein the ring A is thiazoline, thiazole, thiazolidine, thiomorpholine, thiophene, pyrrole, morpholine, azepane, pyridine, pyrazine, furan, 2,3-dihydro-4H-1,4-thiazine, imidazole, or tetrahydrofuran.
[7] The use of [5] or [6], wherein the aforementioned compound is a heterocyclic compound represented by the formula (I) or a salt thereof.
[8] The use of [5], wherein the aforementioned compound is an isothiocyanate compound represented by the formula (II).
[9] A method for enhancing immunity in a mammal, comprising administering an effective amount of at least one kind of compound selected from a heterocyclic compound represented by the formula (I) or a salt thereof, and an isothiocyanate compound represented by the formula (II) to the mammal.
[10] The method of [9] that is a method for enhancing immunity and suppressing inflammation in a mammal.
[11] The method of [9] or [10], wherein the ring A is thiazoline, thiazole, thiazolidine, thiomorpholine, thiophene, pyrrole, morpholine, azepane, pyridine, pyrazine, furan, 2,3-dihydro-4H-1,4-thiazine, imidazole, or tetrahydrofuran.
[12] The method of any one of [9] to [11], wherein the aforementioned compound is a heterocyclic compound represented by the formula (I) or a salt thereof.
[13] The method of [9] or [10], wherein the aforementioned compound is an isothiocyanate compound represented by the formula (II).
[14] At least one kind of compound selected from a heterocyclic compound represented by the formula (I) or a salt thereof, and an isothiocyanate compound represented by the formula (II) for use in enhancing immunity.
[15] The compound of [14] for use in enhancing immunity and suppressing inflammation.
[16] The compound for use according to [14] or [15], wherein the ring A is thiazoline, thiazole, thiazolidine, thiomorpholine, thiophene, pyrrole, morpholine, azepane, pyridine, pyrazine, furan, 2,3-dihydro-4H-1,4-thiazine, imidazole, or tetrahydrofuran.
[17] The compound for use according to any one of [14] to [16], which is a heterocyclic compound represented by the formula (I) or a salt thereof.
[18] The compound for use according to [14] or [15], which is an isothiocyanate compound represented by the formula (II).

### Advantageous Effects of Invention

According to the present invention, an anti-inflammatory type immune-enhancing agent that simultaneously induces an anti-inflammatory action and an immune-enhancing action is provided. The anti-inflammatory type immune-enhancing agent of the present invention can be used to enhance immunity in the prophylaxis or treatment of infections, sepsis, or cancer.

### BRIEF DESCRIPTION OF DRAWINGS

FIG. 1 A-C: The results of classification of peripheral blood mononuclear cells (PBMC) by single cell RNAseq (scRNAseq) 16 hr after intraperitoneal injection of saline (control) or thiazoline-related fear odor (2-methyl-2-thiazoline; 2MT) are shown. A shows the results under control conditions, B shows the results under 2MT administration conditions, and C shows the merge of the results under control and under 2MT conditions. D: The ratios of cells annotated as monocytes and dendritic cells under A and B conditions are shown.

FIG. 2 shows the analysis results of changes in gene expression under control conditions and 2MT administration conditions for each cell type annotated in the scRNAseq analysis of FIG. 1.

FIG. 3 shows the analysis results of the presence ratio of neutrophils and monocytes in peripheral blood by flow cytometry method under conditions of intraperitoneal administration of thiazoline-related fear odor (2MT) or presentation of the odor, and conditions of corticosterone administration.

FIG. 4 shows the measurement results of the number of neutrophils in the peripheral blood 16 hr after intraperitoneal injection of saline (control) or respective compounds in Example 4.

FIG. 5 shows the chemical structures of the compounds used in Example 4.

FIG. 6 shows the measurement results of the number of neutrophils in the peripheral blood 16 hr after intraperitoneal injection of saline (control) or respective compounds, and the chemical structures of the compounds used in Example 5.

FIG. 7 shows the measurement results of the number of monocytes in the peripheral blood 16 hr after intraperitoneal injection of saline (control) or respective compounds, and the chemical structures of the compounds used in Example 6.

FIG. 8 shows the measurement results of the number of monocytes in the peripheral blood 16 hr after intraperitoneal injection of saline (control) or allyl isothiocyanate, and the chemical structure of the compound used in Example 7.

FIG. 9 shows the measurement results of the number of basophils in the peripheral blood 16 hr after intraperitoneal injection of saline (control) or respective compounds, and the chemical structures of the compounds used in Example 8.

FIG. 10 shows the measurement results of the number of lymphocytes in the peripheral blood 16 hr after intraperitoneal injection of saline (control) or respective compounds, and the chemical structures of the compounds used in Example 9.

FIG. 11 shows the measurement results of the ratio of monocytes in the whole blood cells 16 hr after intraperitoneal injection of saline (control) or respective compounds in Example 10.

FIG. 12 shows the chemical structures of the compounds used in Example 10.

FIG. 13 shows the measurement results of the ratio of monocytes in the whole blood cells 16 hr after intraperitoneal injection of saline (control) or allyl isothiocyanate, and the chemical structure of the compound used in Example 11.

FIG. 14 shows the measurement results of the ratio of neutrophils in the whole blood cells 16 hr after intraperitoneal injection of saline (control) or respective compounds in Example 12.

FIG. 15 shows the chemical structures of the compounds used in Example 12.

FIG. 16 shows the measurement results of the ratio of neutrophils in the whole blood cells 16 hr after intraperitoneal injection of saline (control) or respective compounds, and the chemical structures of the compounds used in Example 13.

FIG. 17 shows the measurement results of the ratio of basophils in the whole blood cells 16 hr after intraperitoneal injection of saline (control) or respective compounds in Example 14.

FIG. 18 shows the chemical structures of the compounds used in Example 14.

FIG. 19 shows the measurement results of the ratio of lymphocytes in the whole blood cells 16 hr after intraperitoneal injection of saline (control) or respective compounds, and the chemical structures of the compounds used in Example 15.

### DESCRIPTION OF EMBODIMENTS

The ring A in the formula (I) is a 5- to 7-membered heterocycle containing 1 or 2 hetero atoms selected from a nitrogen atom, an optionally oxidized sulfur atom and an oxygen atom. The ring A is preferably a 5- to 7-membered heterocycle containing 1 or 2 hetero atoms selected from a nitrogen atom and an optionally oxidized sulfur atom. The ring A is more preferably a 5- to 7-membered heterocycle containing a nitrogen atom and an optionally oxidized sulfur atom. The number of members of ring A is more preferably 5 or 6.

Examples of the aforementioned heterocycle includes, but are not limited to, pyrrole, pyridine, pyridazine, pyrimidine, pyrazine, piperazine, pyrrolidine, hexahydropyridazine, imidazole, imidazolidine, piperidine, thiophene, thiolane, tetrahydro-2H-thiopyran, thiazoline (e.g., 2-thiazoline, 3-thiazoline, 4-thiazoline), thiazole, thiazolidine, isothiazole, isothiazoline, thiomorpholine, thiadiazoline, thiadiazole, thiadiazolidine, 1,3-thiazinane, 5,6-dihydro-4H-1,3-thiazine, 2,3-dihydro-4H-1,4-thiazine, furan, 2H-pyran, 4H-pyran, oxazole, isoxazole, morpholine, oxazoline, azepane, tetrahydrofuran, and the like. It is preferably thiazoline, thiazole, thiazolidine, thiomorpholine, thiophene, pyrrole, morpholine, azepane, pyridine, pyrazine, furan, 2,3-dihydro-4H-1,4-thiazine, imidazole, or tetrahydrofuran, more preferably thiazoline (e.g., 2-thiazoline, 3-thiazoline, 4-thiazoline), thiazole, thiazolidine, thiomorpholine, thiophene, pyrrole, morpholine, azepane, pyridine, pyrazine, furan, 2,3-dihydro-4H-1,4-thiazine, or imidazole, further preferably thiazoline (e.g., 2-thiazoline), thiazole, thiazolidine, thiomorpholine, thiophene, or 2,3-dihydro-4H-1,4-thiazine.

The “halogen atom” used here is preferably selected from a fluorine atom, a chlorine atom, a bromine atom, and an iodine atom.

The “C₁₋₆ alkyl group” used here (when used as a group or a part of a group) is a straight chain or branched chain alkyl group having 1 to 6 carbon atoms. Examples of the C₁₋₆ alkyl group include, but are not limited to, methyl group, ethyl group, propyl group, isopropyl group, butyl group, 1-methylpropyl group (sec-butyl group), 2-methylpropyl group (isobutyl group), tert-butyl group, pentyl group, 1-methylbutyl group, 2-methylbutyl group, 3-methylbutyl group, 1,1-dimethylpropyl group, 2,2-dimethylpropyl group, 1,2-dimethylpropyl group, 1-ethylpropyl group, hexyl group, 1-methylpentyl group, 2-methylpentyl group, 3-methylpentyl group, 4-methylpentyl group, 1,1-dimethylbutyl group, 2,2-dimethylbutyl group, 3,3-dimethylbutyl group, 1,2-dimethylbutyl group, 1,3-dimethylbutyl group, 2,3-dimethylbutyl group, 1-ethylbutyl group, 2-ethylbutyl group, 1-ethyl-2-methylpropyl group, and the like. Preferable examples of the C₁₋₆ alkyl group include C₁₋₄ alkyl group (straight chain or branched chain alkyl group having 1 to 4 carbon atoms). It is further preferably methyl group, ethyl group, propyl group, isopropyl group, butyl group, isobutyl group, or sec-butyl group, and particularly preferably methyl group.

The “C₁₋₅ haloalkyl group” used here means a C₁₋₆ alkyl group substituted by 1 to 5 halogeno groups. When two or more halogeno groups are present, the kind of respective halogeno groups may be the same or different. As the halogeno group, a fluoro group, a chloro group, a bromo group, and the like can be mentioned. Examples of the C₁₋₆ haloalkyl group include, but are not limited to, fluoromethyl group, difluoromethyl group, trifluoromethyl group, chlorodifluoromethyl group, 1-fluoroethyl group, 2-fluoroethyl group, 2-chloroethyl group, 2-bromoethyl group, 1,1-difluoroethyl group, 1,2-difluoroethyl group, 2,2,2-trifluoroethyl group, 1,1,2,2-tetrafluoroethyl group, 1,1,2,2,2-pentafluoroethyl group, 1-fluoropropyl group, 1,1-difluoropropyl group, 2,2-difluoropropyl group, 3-fluoropropyl group, 3,3,3-trifluoropropyl group, 4-fluorobutyl group, 4,4,4-trifluorobutyl group, 5-fluoropentyl group, 5,5,5-trifluoropentyl group, 6-fluorohexyl group, 6,6,6-trifluorohexyl group, and the like.

The “C₂₋₆ alkenyl group” used here (when used as a group or a part of a group) means a straight chain or branched chain alkenyl group having 2 to 6 carbon atoms. Examples of the C₂₋₆ alkenyl group include, but are not limited to, vinyl group, allyl group, prop-1-enyl group, but-1-en-1-yl group, but-2-en-1-yl group, pent-4-en-1-yl group, 2-methylallyl group, and the like.

The “C₁₋₆ alkoxy group” used here (when used as a group or a part of a group) means a straight chain or branched chain alkoxy group having 1 to 6 carbon atoms. Examples of the C₁₋₆ alkoxy group include, but are not limited to, methoxy group, ethoxy group, propoxy group, isopropoxy group, butoxy group, 1-methylpropoxy group, 2-methylpropoxy group, tert-butoxy group, pentyloxy group, 1-methylbutoxy group, 2-methylbutoxy group, 3-methylbutoxy group, 1,1-dimethylpropoxy group, 2,2-dimethylpropoxy group, 1,2-dimethylpropoxy group, 1-ethylpropoxy group, hexyloxy group, and the like.

The “C₁₋₆ alkylthio group” used here means an —SH group substituted by a C₁₋₆ alkyl group. Examples of the C₁₋₆ alkylthio group include, but are not limited to, methylthio group, ethylthio group, propylthio group, butylthio group, and the like.

The “C₂₋₆ alkenylthio group” used here means an —SH group substituted by C₂₋₆ alkenyl. Examples of the C₂₋₆ alkenylthio group include, but are not limited to, vinylthio group, allylthio group, prop-1-enylthio group, but-1-en-1-ylthio group, but-2-en-1-ylthio group, pent-4-en-1-ylthio group, 2-methylallylthio group, and the like.

The “C₁₋₆ alkyl-carbonyl group” used here means a carbonyl group bonded to a C₁₋₆ alkyl group. Examples of the C₁₋₆ alkyl-carbonyl group include, but are not limited to, acetyl group, propionyl group, butyryl group, isobutyryl group, valeryl group, hexanoyl group, and the like.

The “C₁₋₆ alkoxycarbonyl group” used here means a carbonyl group bonded to a C₁₋₆ alkoxy group. Examples of the C₁₋₆ alkoxycarbonyl group include, but are not limited to, methoxycarbonyl group, ethoxycarbonyl group, propoxycarbonyl group, isopropoxycarbonyl group, butoxycarbonyl group, and the like.

The “C₆₋₁₀ aryl group” used here means an aromatic hydrocarbon group having 6 to 10 carbon atoms. Examples of the C₆₋₁₀ aryl group include, but are not limited to, phenyl group, naphthyl group (1-naphthyl group, 2-naphthyl group), and the like.

The “C₁₋₆ alkylthio-C₁₋₆ alkyl group” used here means a “C₁₋₆ alkyl group” bonded to a “C₁₋₆ alkylthio group”. Examples of the C₁₋₆ alkylthio-C₁₋₆ alkyl group include, but are not limited to, methylthiomethyl group, 2-(methylthio)ethyl group, 3-(methylthio)propyl group, ethylthiomethyl group, 2-(ethylthio)ethyl group, and the like.

The “C₆₋₁₀ aryl-C₁₋₆ alkyl group” used here means a “C₁₋₆ alkyl group” bonded to a “C₆₋₁₀ aryl group”. Examples of the C₆₋₁₀ aryl-C₁₋₆ alkyl group include, but are not limited to, benzyl group, 2-phenylethyl group and the like.

The “5- or 6-membered heteroaryl group” used here means a 5- or 6-membered heteroaryl group containing at least 1 (preferably 1 to 3, more preferably 1 or 2) hetero atom selected from nitrogen atom, optionally oxidized sulfur atom and oxygen atom. The 5- or 6-membered heteroaryl group is preferably a 5- or 6-membered heteroaryl group containing 1 or 2 hetero atoms selected from nitrogen atom and optionally oxidized sulfur atom.

Examples of the 5- or 6-membered heteroaryl group include, but are not limited to, pyrrolyl group, pyridyl group, pyridazinyl group, pyrimidinyl group, pyrazinyl group, imidazolyl group, thienyl group, thiazolyl group, isothiazolyl group, thiadiazolyl group, furyl group, oxazolyl group, isoxazolyl group, and the like. Preferably, it is pyridyl group, thienyl group, and the like.

The “5- or 6-membered heteroaryl-C₁₋₆ alkyl group” used here means a “C₁₋₆ alkyl group” bonded to a “5- or 6-membered heteroaryl group”. Examples of the 5- or 6-membered heteroaryl-C₁₋₆ alkyl group include, but are not limited to, furfuryl group and the like.

The “5- or 6-membered heteroaryl-C₁₋₆ alkylthio group” used here means a “C₁₋₆ alkylthio group” bonded to a “5- or 6-membered heteroaryl group”. Examples of the 5- or 6-membered heteroaryl-C₁₋₆ alkylthio group include, but are not limited to, furfurylthio group and the like.

The term “oxo group” used here (when used as a group or a part of a group) shows an ═O group.

The term “optionally oxidized sulfur atom” used here means S, SO, or SO₂.

The “5- to 10-membered ring” of the “optionally substituted 5- to 10-membered ring”, which is formed by R¹ and R² bonded to each other, means a 5- to 10-membered ring optionally having at least 1 (preferably 1 to 3, more preferably 1 or 2) hetero atoms selected from a nitrogen atom, an optionally oxidized sulfur atom and an oxygen atom. Examples of the aforementioned 5- to 10-membered ring include benzene ring, tetrahydropyrimidine ring, decahydronaphthalene ring, and the like. The aforementioned 5- to 10-membered ring may be substituted, and examples of the substituent include 1 to 4 (preferably 1 or 2) substituents selected from C₁₋₆ alkyl group, C₁₋₆ alkoxy group, halogen atom, amino group, —SH, C₁₋₆ alkylthio group, C₂₋₆ alkenylthio group, C₁₋₆ alkyl-carbonyl group, formyl group, C₁₋₆ alkoxycarbonyl group, oxo group, and the like. As the substituent, 1 to 4 substituents selected from C₁₋₆ alkyl group (e.g., methyl), C₁₋₆ alkoxy group (e.g., methoxy), and oxo group are preferred.

The “optionally substituted 5- to 10-membered ring” formed by R¹ and R² bonded to each other is preferably an “optionally substituted 5- or 6-membered ring”. The “5- or 6-membered ring” of the “optionally substituted 5- or 6-membered ring” formed by R¹ and R² bonded to each other means a 5- or 6-membered ring optionally containing at least 1 (preferably 1 to 3, more preferably 1 or 2) hetero atom selected from nitrogen atom, optionally oxidized sulfur atom and oxygen atom. Examples of the aforementioned 5- or 6-membered ring include benzene ring, tetrahydropyrimidine ring, and the like. The aforementioned 5- or 6-membered ring may be substituted, and examples of the substituent include 1 to 4 (preferably 1 or 2) substituents selected from C₁₋₆ alkyl group, halogen atom, amino group, —SH, C₁₋₆ alkylthio group, C₂₋₆ alkenylthio group, C₁₋₆ alkyl-carbonyl group, formyl group, C₁₋₆ alkoxycarbonyl group, oxo group, and the like. The substituent is preferably 1 to 4 substituents selected from C₁₋₆ alkyl group (e.g., methyl) and oxo group.

In the formula (I), preferably, R¹, R², R³ and R⁴ are each independently hydrogen atom, C₁₋₆ alkyl group (e.g., methyl, ethyl, propyl, isopropyl, butyl, isobutyl, sec-butyl, tert-butyl, hexyl), C₁₋₆ alkoxy group (e.g., methoxy, ethoxy), halogen atom (e.g., chlorine atom), amino group, —SH, C₁₋₆ alkylthio group (e.g., methylthio), C₂₋₆ alkenylthio group (e.g., allylthio), C₁₋₆ alkyl-carbonyl group (e.g., acetyl, propionyl), formyl group, C₆₋₁₀ aryl group (e.g., phenyl), 5- or 6-membered heteroaryl group (e.g., thienyl), 5- or 6-membered heteroaryl-C₁₋₆ alkyl group (e.g., furfuryl), 5- or 6-membered heteroaryl-C₁₋₆ alkylthio group (e.g., furfurylthio), or oxo group; R¹ and R² are optionally bonded to each other to form an optionally substituted 5- to 10-membered ring (e.g., benzene ring, tetrahydropyrimidine ring, decahydronaphthalene ring).

In the formula (I), preferably, R¹, R², R³ and R⁴ are each independently hydrogen atom, C₁₋₆ alkyl group (e.g., methyl, ethyl, propyl, isopropyl, butyl, isobutyl, sec-butyl, tert-butyl, hexyl), halogen atom (e.g., chlorine atom), amino group, —SH, C₁₋₆ alkylthio group (e.g., methylthio), C₂₋₆ alkenylthio group (e.g., allylthio), C₁₋₆ alkyl-carbonyl group (e.g., acetyl, propionyl), formyl group, C₆₋₁₀ aryl group (e.g., phenyl), 5- or 6-membered heteroaryl group (e.g., thienyl), or oxo group; and R¹ and R² are optionally bonded to each other to form an optionally substituted 5- or 6-membered ring (e.g., benzene ring, tetrahydropyrimidine ring).

In the formula (I), when n=1 or 2, it is preferable that at least one of R¹, R², R³ and R⁴ is not a hydrogen atom. In the formula (I), when n=0, it is preferable that at least one of R¹, R² and R³ is not a hydrogen atom.

In the formula (II), preferably, R⁵ is C₁₋₆ alkyl group (e.g., isopropyl, hexyl), C₁₋₆ haloalkyl group, C₂₋₆ alkenyl group (e.g., allyl, methallyl, 4-pentenyl), C₁₋₆ alkylthio-C₁₋₆ alkyl group (e.g., 3-(methylthio)propyl), or C₆₋₁₀ aryl-C₁₋₆ alkyl group (e.g., benzyl).

Examples of the preferable heterocyclic compound of the formula (I) used as the active ingredient in the present invention include, but are not limited to, the following compounds:
2-methyl-2-thiazoline (2MT)
4-ethyl-2-methyl-thiazoline
2-mercaptothiazoline
2-(methylthio)-2-thiazoline
4-(methylthio)-2-thiazoline
2-isobutyl-4,5-dimethyl-3-thiazoline
2-ethoxythiazole
5-acetyl-2,4-dimethylthiazole
4,5-dimethylthiazole
5-methylthiazole
4-tert-butyl-2-methylthiazole
2-(methylthio)benzothiazole
2-methylthiomorpholine
2H-1,4-benzothiazin-3(4H)one
thiazolidine-2,4-dione
3-acetyl-2,5-dimethylthiophene
2,5-dimethylthiophene
2-ethylthiophene
2-hexylthiophene
3-chlorothiophene
benzothiophene
2-furfurylthio-3-methylpyrazine
2,3-diethylpyrazine
2-acetylpyrrole
2-acetyl-1-methylpyrrole
3-acetylpyridine
4-acetylpyridine
azepane
6-methoxyquinoline
morpholine
2-(allylthio)-2-thiazoline
4-phenyl-thiomorpholine-1,1-dioxide
2-acetyl-3,5-dimethylpyrazine
2-methyl-3-tetrahydrofuranthiol
2,6-lutidine
2-acetyl-5-methylfuran
2,5-dimethylfuran
2,5-dimethyltetrahydrofuran
2-methylfuran
2,3-dimethylbenzofuran
2-acetyl-6-methylpyridine
2,3-dimethylpyridine
(−)-ambroxide
thiomorpholine
2,3-dimethylthiomorpholine
2-acetyl-3-ethylpyrazine
2-methyl-3-methylthiopyrazine, 2-methyl-5-methylthiopyrazine,
2-methyl-6-methylthiopyrazine, or a mixture of these isomers
2-methylindole
thiazole
2-methylthiazole
2-isobutylthiazole
2,4,5-trimethylthiazole
2-chlorothiazole
2-acetylthiazole
2-isopropyl-4-methylthiazole
2,2-dimethylthiazolidine
2-propionylthiophene
2-methylthiophene
1-furfurylpyrrole
2-ethylpyrrole
2,4-dimethylpyrrole
3-methylpyrrole
3-methylthiophene
3-acetyl-2,5-dimethylfuran
2-ethylfuran
5-formylthiazole.

Examples of the preferable isothiocyanate compound of the formula (II) used as the active ingredient in the present invention include, but are not limited to, the following compounds:
allyl isothiocyanate
isopropyl isothiocyanate
methallyl isothiocyanate
4-pentenyl isothiocyanate
3-(methylthio)propyl isothiocyanate
hexyl isothiocyanate
benzyl isothiocyanate.

In the present invention, a heterocyclic compound of the formula (I) and an isothiocyanate compound of the formula (II) used as the active ingredients include substances generally known as reagents, commercially available ones can be utilized, and they can be obtained by a method known per se. Use of the heterocyclic compound of the formula (I) and the isothiocyanate compound of the formula (II) as anti-inflammatory type immune-enhancing agent has not been disclosed or suggested to date.

Preferable examples of the heterocyclic compound represented by the formula (I) include compounds represented by the following formulas (A) to (E) or salts thereof.

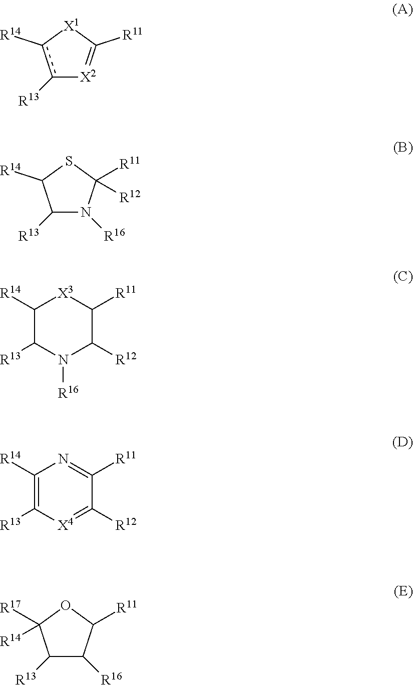

wherein
X¹ is S, O, or N(R¹⁶);
X² is N or CR¹²;
X³ is S, SO₂, O, or —(CH₂)₂—;
X⁴ is N or CR¹⁵;

[symbol]

is a single bond or a double bond;
R¹¹, R¹², R¹³, R¹⁴, R¹⁵, R¹⁶, and R¹⁷ are each independently a hydrogen atom, a C₁₋₆ alkyl group, a C₁₋₆ alkoxy group, a halogen atom, an amino group, —SH, a C₁₋₆ alkylthio group, a C₂₋₆ alkenylthio group, a C₁₋₆ alkyl-carbonyl group, a formyl group, a C₆₋₁₀ aryl group, a C₁₋₆ alkoxycarbonyl group, a 5- or 6-membered heteroaryl group, a 5- or 6-membered heteroaryl-C₁₋₆ alkyl group, a 5- or 6-membered heteroaryl-C₁₋₆ alkylthio group, or an oxo group;
R¹³ and R¹⁴ are optionally bonded to each other to form a benzene ring optionally substituted by 1 to 4 C₁₋₆ alkoxy groups, a tetrahydropyrimidine ring optionally substituted by 1 to 4 substituents selected from a C₁₋₆ alkyl group and an oxo group, or a decahydronaphthalene ring optionally substituted by 1 to 4 C₁₋₆ alkyl groups;
provided that, in the formula (A), R¹¹ and R¹² are not oxo groups; in the formula (A), when

[symbol]

is a double bond, R¹³ and R¹⁴ are not oxo groups;
in the formula (D), R¹¹, R¹², R¹³, R¹⁴ and R¹⁵ are not oxo groups, and in the formula (B), R¹¹ and R¹² may together form an oxo group.

In the formulas (A) to (E), preferably, R¹¹, R¹², R¹³, R¹⁴, R¹⁵, R¹⁶, and R¹⁷ are each independently a hydrogen atom, a C₁₋₆ alkyl group (e.g., methyl, ethyl, propyl, isopropyl, butyl, isobutyl, sec-butyl, tert-butyl, hexyl), a C₁₋₆ alkoxy group (e.g., methoxy, ethoxy), a halogen atom (e.g., chlorine atom), an amino group, —SH, a C₁₋₆ alkylthio group (e.g., methylthio), a C₂₋₆ alkenylthio group (e.g., allylthio), a C₁₋₆ alkyl-carbonyl group (e.g., acetyl, propionyl), a formyl group, a C₆₋₁₀ aryl group (e.g., phenyl), a 5- or 6-membered heteroaryl group (e.g., thienyl), a 5- or 6-membered heteroaryl-C₁₋₆ alkyl group (e.g., furfuryl), a 5- or 6-membered heteroaryl-C₁₋₆ alkylthio group (e.g., furfurylthio), or an oxo group;

R¹³ and R¹⁴ are optionally bonded to each other to form a benzene ring optionally substituted by 1 to 4 C₁₋₆ alkoxy groups, a tetrahydropyrimidine ring optionally substituted by 1 to 4 substituents selected from a C₁₋₆ alkyl group and an oxo group, or a decahydronaphthalene ring optionally substituted by 1 to 4 C₁₋₆ alkyl groups.

The salt of the compound of the present invention may be a pharmaceutically acceptable salt. For example, alkali metal salts such as sodium salt and potassium salt; alkaline earth metal salts such as magnesium salt and calcium salt; ammonium salts such as dimethylammonium salt and triethylammonium salt; inorganic acid salts such as hydrochloride, perchlorate, sulfate and nitrate; organic acid salts such as acetate and methanesulfonate; and the like can be mentioned.

The anti-inflammatory type immune-enhancing agent disclosed by the present invention can be utilized as a prophylactic or therapeutic drug for infections, sepsis, or cancer. The anti-inflammatory type immune-enhancing agent of the present invention can be used to enhance immunity in the prophylaxis or treatment of infections, sepsis, or cancer. The “anti-inflammatory type immune-enhancing agent” means a medicament that shows an anti-inflammatory action and an immune-enhancing action.

Examples of the infections include infections with bacteria, viruses, fungi, and the like.

Examples of the cancer include solid cancers such as head and neck cancer, esophageal cancer, gastric cancer, colorectal cancer, colon cancer, rectal cancer, liver cancer, pancreatic cancer, lung cancer (small cell lung cancer, non-small cell lung cancer), breast cancer, uterine body cancer, cervical cancer, ovarian cancer, prostate cancer, kidney cancer, bladder cancer, oral cancer, gall bladder cancer, bile duct cancer, malignant melanoma, mesothelioma, and the like; and hematopoietic tumors such as acute myelogenous leukemia, acute lymphocytic leukemia, chronic myelogenous leukemia, chronic lymphocytic leukemia, malignant lymphoma, multiple myeloma and the like.

A heterocyclic compound of the formula (I) or a salt thereof, or an isothiocyanate compound of the formula (II) (hereinafter also to be referred to as the compound of the present invention) can be administered to animals, including a human who has or may develop infections, sepsis or cancer, for the purpose of preventing the development of injury or alleviating the symptoms. A gas derived from the compound of the present invention and developed at a concentration of 0.1 to 100,000 ppm can be inhaled through the nasal cavity or lung using a gas mask or a device having a similar function. Alternatively, the compound of the present invention can be administered orally at a dose of 1 μg/kg to 5,000 mg/kg. Alternatively, the compound of the present invention can be intracorporeally injected at a dose of 1 μg/kg to 5,000 mg/kg by a method such as intradermal injection, subcutaneous injection, intramuscular injection, intravenous injection, intraarterial injection, intraspinal injection, intraperitoneal injection, and the like. The administration frequency may be single-dose administration, or continuous administration at regular intervals, or continuous administration at different time intervals. The animal to be the subject of administration may be, for example, mammals (human, mouse, rat, hamster, rabbit, cat, dog, bovine, sheep, swine, horse, monkey, and the like).

When the compound of the present invention is used as an anti-inflammatory type immune-enhancing agent (hereinafter to be also referred to as the agent of the present invention), a pharmaceutically acceptable additive can be added as necessary.

Specific examples of the pharmaceutically acceptable additive include, but are not limited to, antioxidant, preservative, colorant, flavoring agent, and diluent, emulsifier, suspending agent, solvent, filler, extending agent, buffering agent, delivery vehicle, diluent, carrier, excipient and/or pharmaceutical adjuvant, and the like.

The dosage form of the agent of the present invention is not particularly limited and, for example, liquid, injection, sustained-release preparation, and the like can be mentioned. The solvent to be used for formulating the agent of the present invention in the above-mentioned dosage form may be aqueous or non-aqueous.

An injection can be prepared by a method well known in the pertinent field. For example, an injection can be prepared by dissolving in an appropriate solvent (saline, buffer such as PBS, sterile water, and the like), sterilizing by filtration with a filter or the like, and then filling in an aseptic container (e.g., ampoule and the like). The injection may contain a conventionally-used pharmacological carrier as necessary. An administration method using a non-invasive catheter can also be adopted. The carrier that can be used in the present invention includes neutral buffered saline, saline containing serum albumin, and the like.

The present invention is explained in more detail in the following by referring to Examples. The Examples do not limit the present invention.

### Example 1: Innate Immunocytes that Increase in Peripheral Blood Due to Thiazoline-Related Fear Odor

### Experiment Method

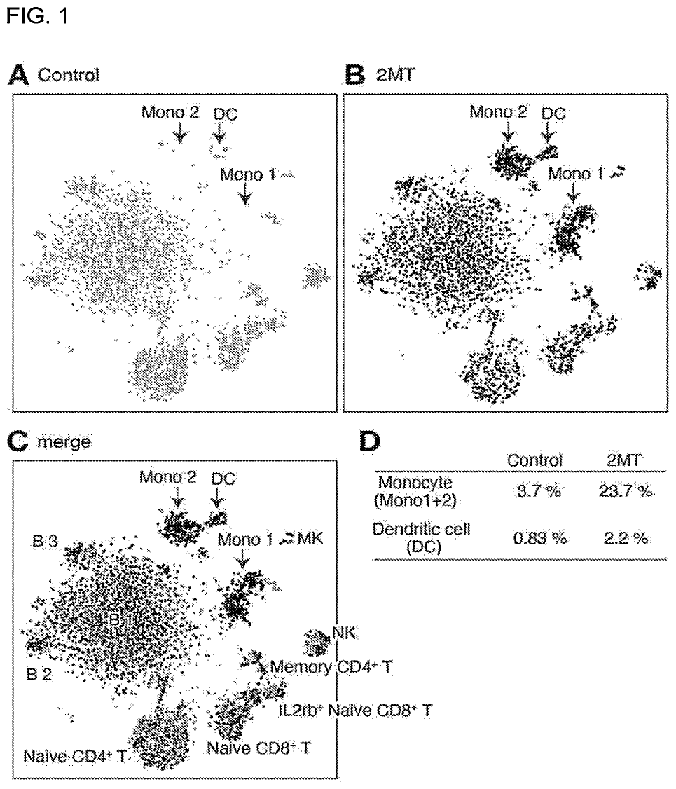

About 3-month-old C57/BL6 mice were intraperitoneally administered with 200 μl of saline (control) or 1% 2MT solution (dissolved in saline). Blood was collected 16 hr later, and peripheral blood mononuclear cells (PBMC) were separated using Ficoll Paque PREMIUM 1.084 (GE Healthcare). A single cell RNA cDNA library was prepared from the separated cells by using the 10× Chromium preparation system, Chromium Single Cell 3′ Library & Gel Bead Kit v3, and sequencing was performed using the illumina HiSeq. The obtained scRNAseq data was analyzed according to the method shown in Non-Patent Literatures 4 and 5. The results of clustering using the gene expression of each cell as an index were visualized by tSNE, and the results are shown in FIG. 1A-C. A is the cell type (gray) present in PBMC of the control mice administered with saline, B is the cell type (black) present in PBMC of the mice subjected to 2MT stimulation, and C shows the results of A and B merge. D shows the cell ratio of monocytes (Monocyte: total of Mono 1 and Mono 2 clusters of A-B) and dendritic cells in PBMC under control conditions and 2MT administration conditions.

### Results

The results are shown in FIG. 1.

It was clarified that 2MT stimulation that induces innate fear emotion drastically increases the numbers of monocytes (Mono 1, Mono 2) and dendritic cells (DC) involved in the enhancement of innate immunity (A-C). Monocytes can be classified into two groups based on the specificity of gene expression, and they were respectively named Mono 1 and Mono 2. In addition to the monocytes and dendritic cells, 3 kinds of B cells (B1, B2, B3), 4 kinds of T cells (Naive CD4⁺ T, Naive CD8⁺ T, Memory CD4⁺ T, IL2rb⁺ Naive CD8⁺ T), and megakaryocytes (MK) were detected.

The percentage of monocytes in PBMC was only 3.7% under control conditions but increased to 23.7% under 2MT stimulation conditions. Dendritic cells were 0.83% under the control conditions but increased to 2.2% by 2MT stimulation (FIG. 1D). From the above results, it was shown that innate fear odor stimulation increases the serum ratio of monocytes and dendritic cells involved in innate immunity.

### Example 2: Inflammation-Suppressing Genes and Immune-Enhancing Genes Showing Potentiated Expression Due to Innate Fear Odor Stimulation

### Experiment Method

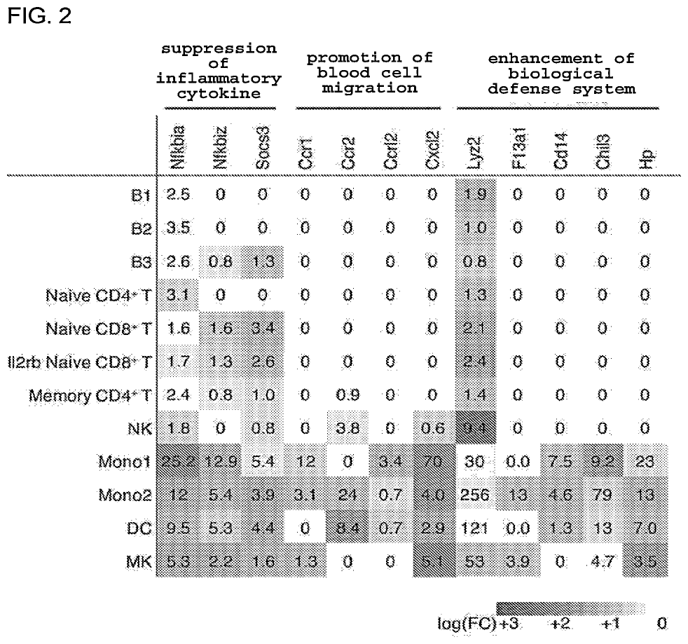

Based on the data of scRNAseq performed in Example 1, the comparison results of the changes in the expression of each gene in the cells present in each cluster between the control conditions and the 2MT administration conditions are shown in FIG. 2. The genes whose expression changed by 2MT stimulation are shown on the top of the table, and the functional categories of the genes are shown above them. The numbers in the table indicate gene expression levels, and the density of shading indicates the degree of increase in gene expression. Darker shading indicates that gene expression increased more.

### Results

The results are shown in FIG. 2.

Compared with the control conditions, 2MT stimulation increased the expression levels of Nfkbia, Nfkbiz, and Socs3, which are suppressors of the NF-κB pathway and JAK-STAT pathway, which promote inflammation by cytokine release. Therefore, 2MT stimulation is considered to suppress inflammation. 2MT stimulation increased the expression levels of Ccr1, Ccr2, Ccr12, and Cxc12, which are genes that promote blood cell migration, and also increased the expression levels of Lyz2, F13a1, Cd14, Chil3, and Hp, which are involved in the enhancement of biological defense response. These changes in gene expression are assumed to increase the number of cells in the blood involved in innate immunity and to enhance immunocompetence. From the above results, it was shown that innate fear stimulation by 2MT enhances innate immunity by promoting the migration of monocytes, neutrophils, and the like into the blood, as well as suppresses inflammatory responses that are normally induced simultaneously with the migration of these, by increasing the expression of cytokine suppressor genes, namely, it induces the critical immune state in which the innate immunity is enhanced and the inflammation is suppressed.

### Example 3: Innate Immunocytes that Increase in Peripheral Blood Due to Intraperitoneal Injection and Odor Stimulation of Innate Fear Odor

### Experiment Method

About 3-month-old C57/BL6 mice were intraperitoneally administered with 200 μl of saline (control) or 1% 2MT solution (dissolved in saline). Blood was collected 16 hr later, and the number of various leukocytes in the peripheral blood was measured by a flow cytometry method by peroxidase staining and a two-angle laser flow cytometry method. The ratios of neutrophils and monocytes in the peripheral blood under respective conditions that were clarified by the measurement are shown.

### Results

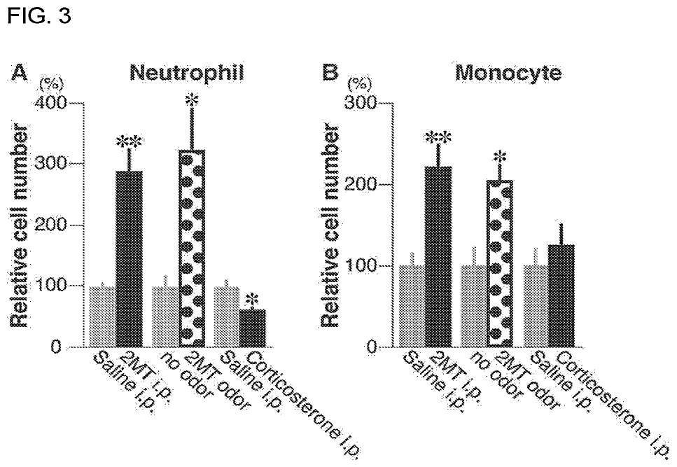

The results are shown in FIG. 3. The vertical axis shows the relative cell number.

A. The number of neutrophils increased in the condition of intraperitoneal injection of 2MT (black bar, 2MT i.p.) as compared with the condition of intraperitoneal injection of saline (Saline i.p.). In addition, the number of neutrophils also increased under the condition with 2MT odor stimulation (dotted bar, 2MT odor) compared to the condition without odor stimulation (no odor). 2MT stimulation increases blood stress hormone (Corticosterone). However, the number of neutrophils decreased under the condition with an intraperitoneal injection of Corticosterone to the same concentration as the level increased by 2MT stimulation (Corticosterone i.p.).

B. The number of monocytes increased in the condition of intraperitoneal injection of 2MT (black bar, 2MT i.p.) as compared with the condition of intraperitoneal injection of saline (Saline i.p.). In addition, the number of monocytes also increased under the condition with 2MT odor stimulation (dotted bar, 2MT odor) compared to the condition without odor stimulation (no odor). 2MT stimulation increases blood stress hormone (Corticosterone). However, the number of monocytes did not increase significantly under the condition with an intraperitoneal injection of Corticosterone to the same concentration as the level increased by 2MT stimulation (Corticosterone i.p.). N=6, Student's t-test, *P<0.05, ** P<0.01

### Example 4

### Experiment Method

About 3-month-old C57/BL6 mice were intraperitoneally injected with 200 μl of saline (control) or 1% solution of respective compounds (dissolved in saline). Blood was collected 16 hr later, and the number of neutrophils in the peripheral blood was measured using Siemens Advia 120.

### Results

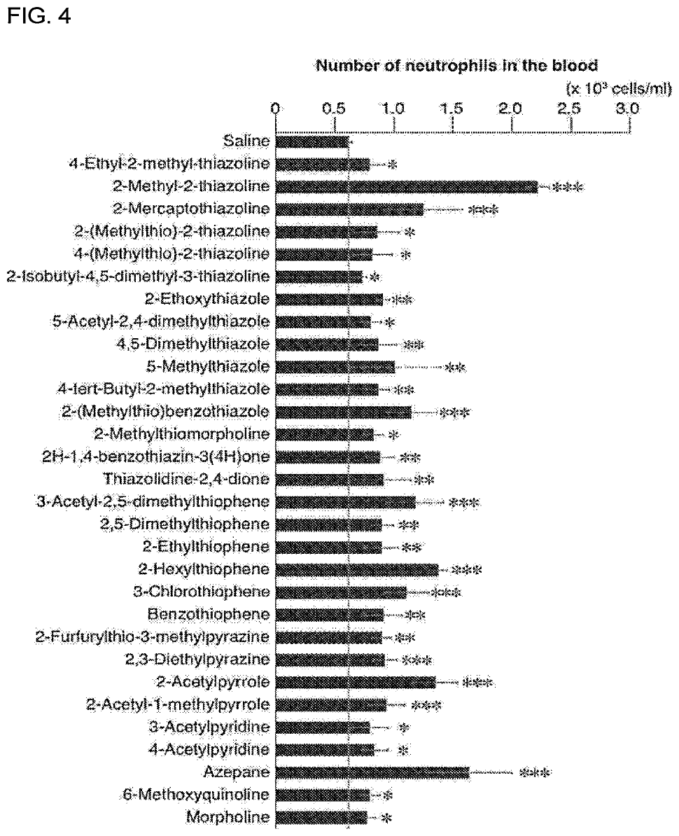

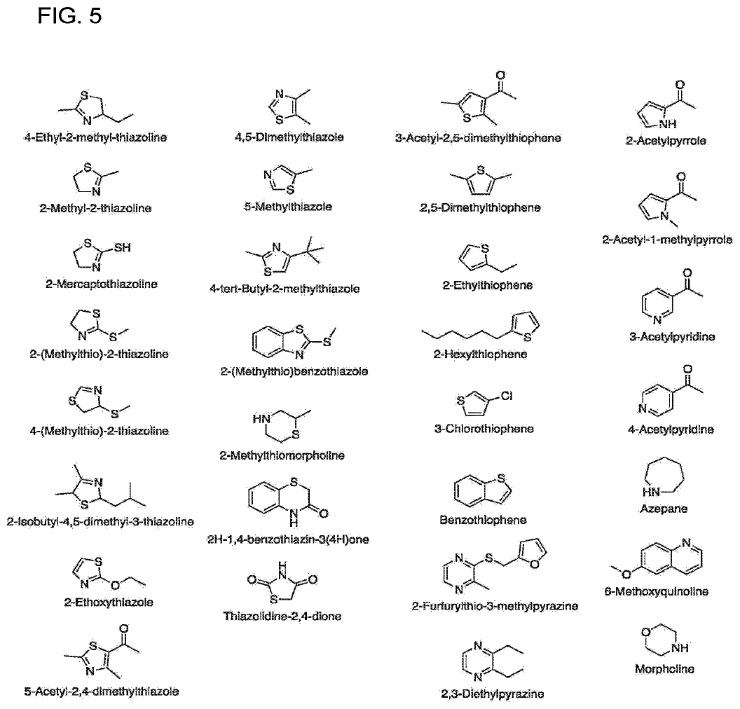

The results are shown in FIG. 4. In addition, the chemical structures of the compounds used are shown in FIG. 5. It was clarified that the following compounds increase neutrophils in the peripheral blood.
4-Ethyl-2-methyl-thiazoline
2-Methyl-2-thiazoline
2-Mercaptothiazoline
2-(Methylthio)-2-thiazoline
4-(Methylthio)-2-thiazoline
2-Isobutyl-4,5-dimethyl-3-thiazoline
2-Ethoxythiazole
5-Acetyl-2,4-dimethylthiazole
4,5-Dimethylthiazole
5-Methylthiazole
4-tert-Butyl-2-methylthiazole
2-(Methylthio)benzothiazole
2-Methylthiomorpholine
2H-1,4-benzothiazin-3(4H)one
Thiazolidine-2,4-dione
3-Acetyl-2,5-dimethylthiophene
2,5-Dimethylthiophene
2-Ethylthiophene
2-Hexylthiophene
3-Chlorothiophene
Benzothiophene
2-Furfurylthio-3-methylpyrazine
2,3-Diethylpyrazine
2-Acetylpyrrole
2-Acetyl-1-methylpyrrole
3-Acetylpyridine
4-Acetylpyridine
Azepane
6-Methoxyquinoline
Morpholine
   n≥5, Student's t-test, * P<0.05, ** P<0.01, *** P<0.001

### Example 5

### Experiment Method

About 3-month-old C57/BL6 mice were intraperitoneally injected with 200 μl of saline (control) or 1% solution of respective compounds (dissolved in saline). Blood was collected 16 hr later, and the number of neutrophils in the peripheral blood was measured using Siemens Advia 120.

### Results

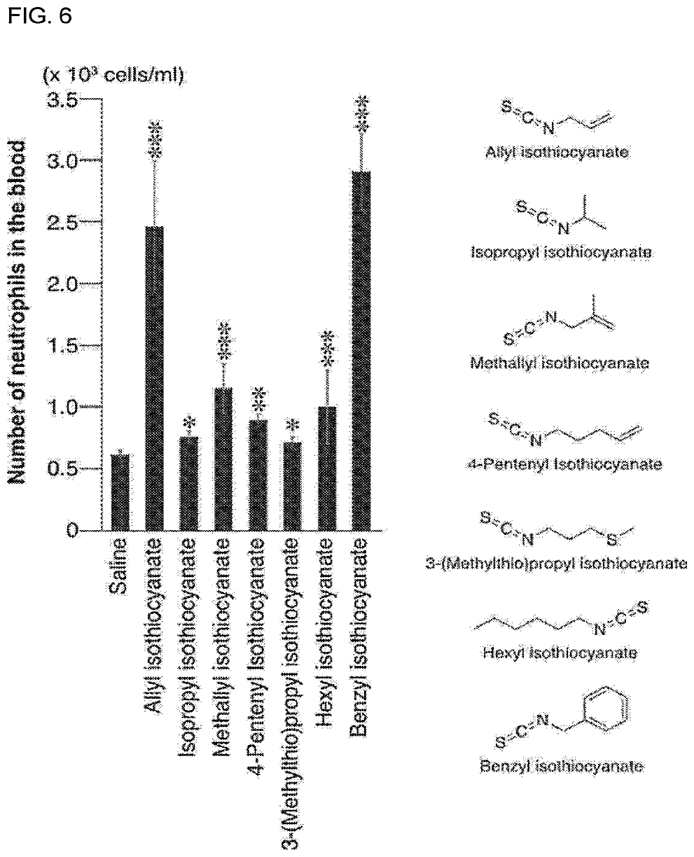

The results and the chemical structures of the compounds used are shown in FIG. 6. It was clarified that the following compounds increase neutrophils in the peripheral blood:
Allyl isothiocyanate
Isopropyl isothiocyanate
Methallyl isothiocyanate
4-Pentenyl Isothiocyanate
3-(Methylthio)propyl isothiocyanate
Hexyl isothiocyanate
Benzyl isothiocyanate
   N≥5, Student's t-test, * P<0.05, ** P<0.01, *** P<0.001

### Example 6

### Experiment Method

About 3-month-old C57/BL6 mice were intraperitoneally injected with 200 μl of saline (control) or 1% solution of respective compounds (dissolved in saline). Blood was collected 16 hr later, and the number of monocytes in the peripheral blood was measured using Siemens Advia 120.

### Results

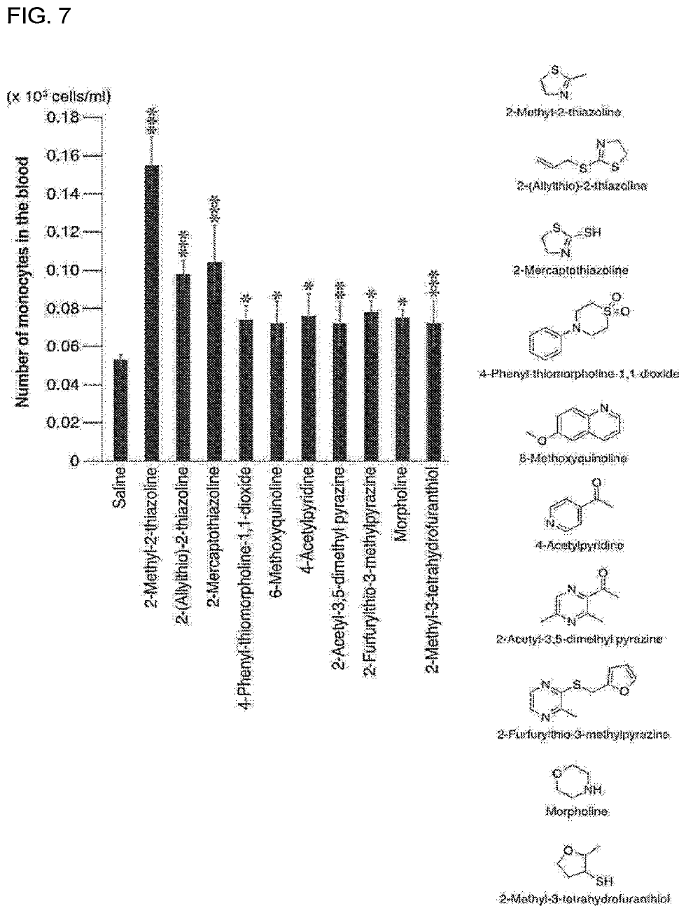

The results and the chemical structures of the compounds used are shown in FIG. 7. It was clarified that the following compounds increase the number of monocytes in the peripheral blood.
2-Methyl-2-thiazoline
2-(Allylthio)-2-thiazoline
2-Mercaptothiazoline
4-Phenyl-thiomorpholine-1,1-dioxide
6-Methoxyquinoline
4-Acetylpyridine
2-Acetyl-3,5-dimethy pyrazine
2-Furfurylthio-3-methylpyrazine
Morpholine
2-Methyl-3-tetrahydrofuranthiol
   N≥5, Student's t-test, * P<0.05, ** P<0.01, *** P<0.001

### Example 7

### Experiment Method

About 3-month-old C57/BL6 mice were intraperitoneally injected with 200 μl of saline (control) or 1% allyl isothiocyanate solution (dissolved in saline). Blood was collected 16 hr later, and the number of monocytes in the peripheral blood was measured using Siemens Advia 120.

### Results

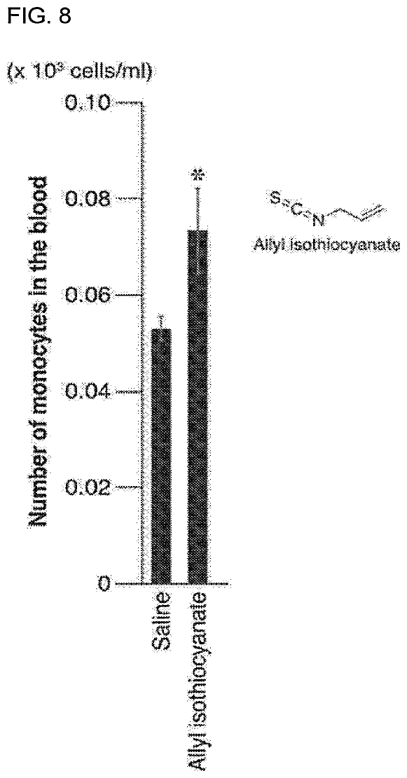

The results are shown in FIG. 8. It was clarified that the administration of allyl isothiocyanate increases the number of monocytes in the peripheral blood. N=6, Student's t-test, * P<0.05

### Example 8

### Experiment Method

About 3-month-old C57/BL6 mice were intraperitoneally injected with 200 μl of saline (control) or 1% solution of respective compounds (dissolved in saline). Blood was collected 16 hr later, and the number of basophils in the peripheral blood was measured using Siemens Advia 120.

### Results

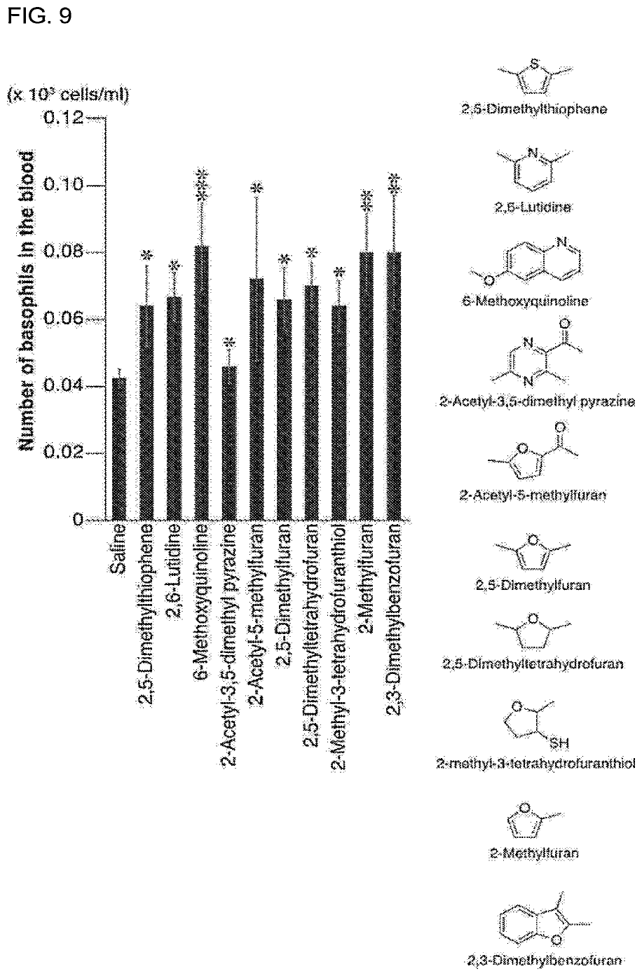

The results and the chemical structures of the compounds used are shown in FIG. 9. It was clarified that the administration of the following compounds increases the number of basophils in the peripheral blood.
2,5-Dimethylthiophene
2,6-Lutidine
6-Methoxyquinoline
2-Acetyl-3,5-dimethy pyrazine
2-Acetyl-5-methylfuran
2,5-Dimethylfuran
2,5-Dimethyltetrahydrofuran
2-Methyl-3-tetrahydrofuranthiol
2-Methylfuran
2,3-Dimethylbenzofuran
   N≥5, Student's t-test, * P<0.05, ** P<0.01, *** P<0.001

### Example 9

### Experiment Method

About 3-month-old C57/BL6 mice were intraperitoneally injected with 200 μl of saline (control) or 1% solution of respective compounds (dissolved in saline). Blood was collected 16 hr later, and the number of lymphocytes in the peripheral blood was measured using Siemens Advia 120.

### Results

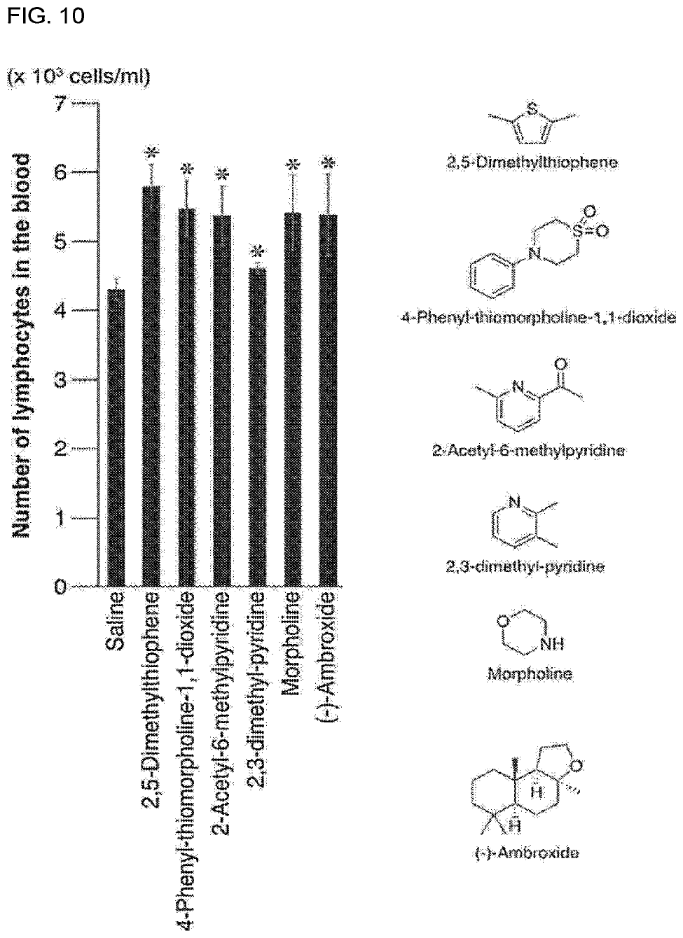

The results and the chemical structures of the compounds used are shown in FIG. 10. It was clarified that the administration of the following compounds increases the number of lymphocytes in the peripheral blood.
2,5-Dimethylthiophene
4-Phenyl-thiomorpholine-1,1-dioxide
2-Acetyl-6-methylpyridine
2,3-dimethylpyridine
Morpholine
(−)-Ambroxide
   N≥5, Student's t-test, * P<0.05

### Example 10

### Experiment Method

About 3-month-old C57/BL6 mice were intraperitoneally injected with 200 μl of saline (control) or 1% solution of respective compounds (dissolved in saline). Blood was collected 16 hr later, and the ratio of monocytes in the whole blood cells was measured using Siemens Advia 120.

### Results

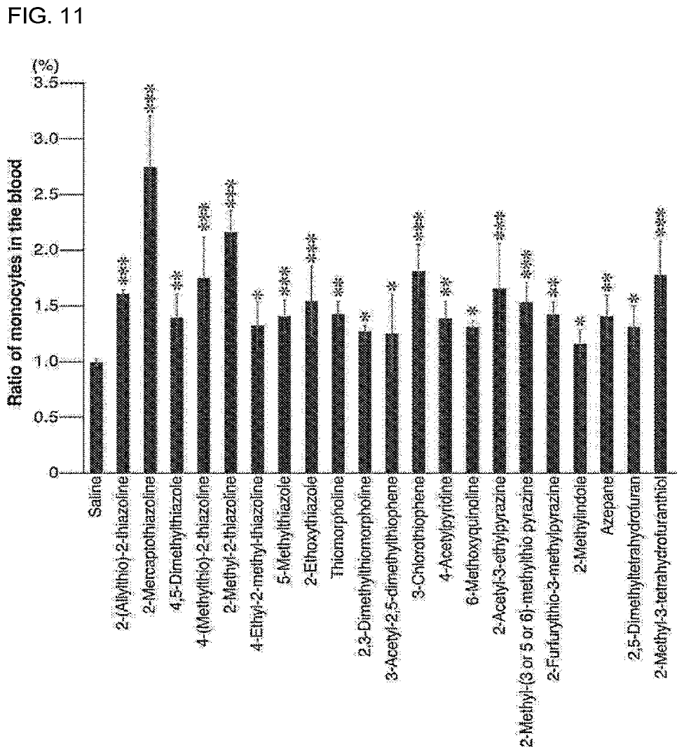

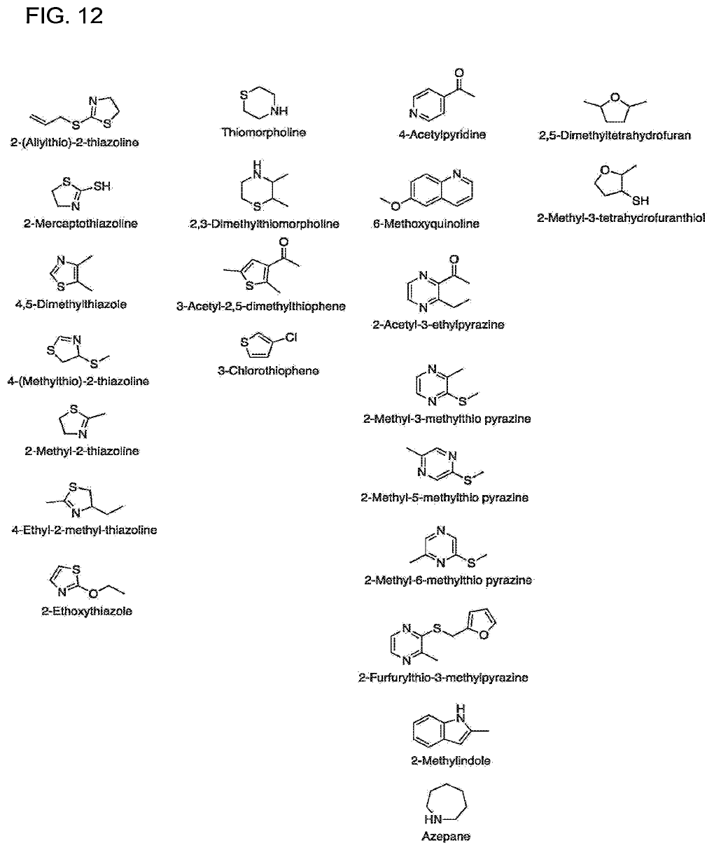

The results are shown in FIG. 11, and the chemical structures of the compounds used are shown in FIG. 12. It was clarified that the administration of the following compounds increases the ratio of monocytes in the whole blood cells.
2-(Allylthio)-2-thiazoline
2-Mercaptothiazoline
4,5-Dimethylthiazole
4-(Methylthio)-2-thiazoline
2-Methyl-2-thiazoline
4-Ethyl-2-methyl-thiazoline
5-Methylthiazole
2-Ethoxythiazole
Thiomorpholine
2,3-Dimethylthiomorpholine
3-Acetyl-2,5-dimethylthiophene
3-Chlorothiophene
4-Acetylpyridine
6-Methoxyquinoline
2-Acetyl-3-ethylpyrazine
2-Methyl-(3 or 5 or 6)-methylthio pyrazine (mixture of isomers)
2-Furfurylthio-3-methylpyrazine
2-Methylindole
Azepane
2,5-Dimethyltetrahydrofuran
2-Methyl-3-tetrahydrofuranthiol N≥5, Student's t-test, * P<0.05, ** P<0.01, *** P<0.001

### Example 11

### Experiment Method

About 3-month-old C57/BL6 mice were intraperitoneally injected with 200 μl of saline (control) or 1% allyl isothiocyanate solution (dissolved in saline). Blood was collected 16 hr later, and the ratio of monocytes in the whole blood cells was measured using Siemens Advia 120.

### Results

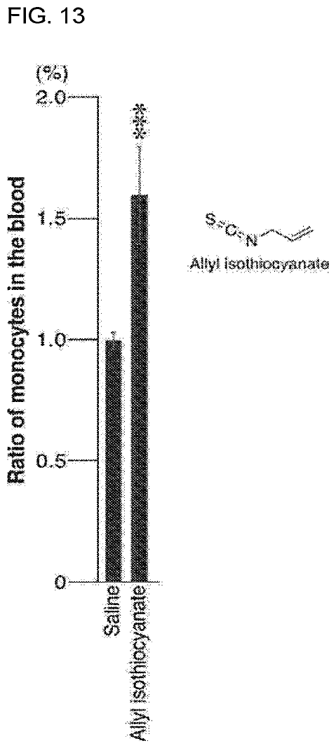

The results and the chemical structure of the compound used are shown in FIG. 13. It was clarified that the administration of allyl isothiocyanate increases the ratio of monocytes in the whole blood cells. N=6, Student's t-test, *** P<0.001

### Example 12

### Experiment Method

About 3-month-old C57/BL6 mice were intraperitoneally injected with 200 μl of saline (control) or 1% solution of respective compounds (dissolved in saline). Blood was collected 16 hr later, and the ratio of neutrophils in the whole blood cells was measured using Siemens Advia 120.

### Results

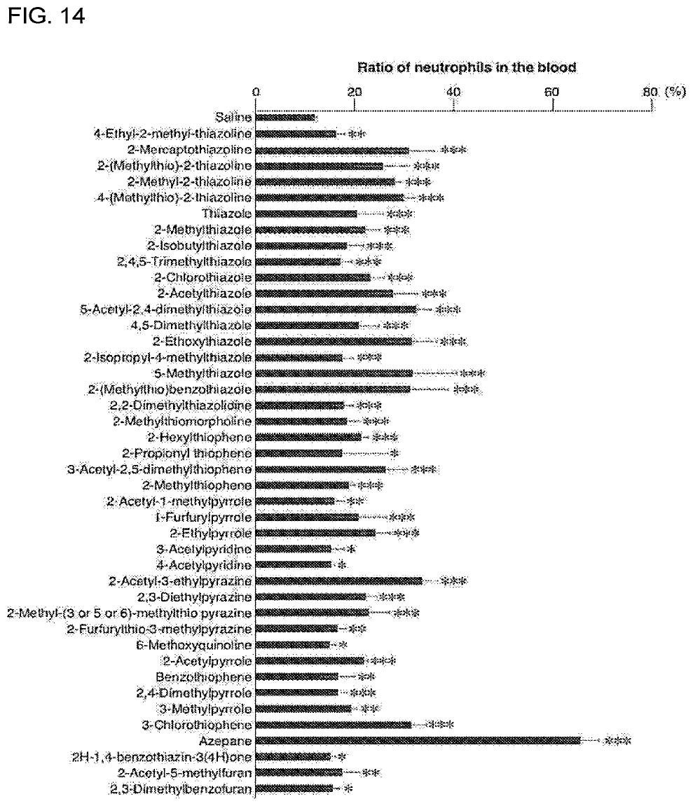

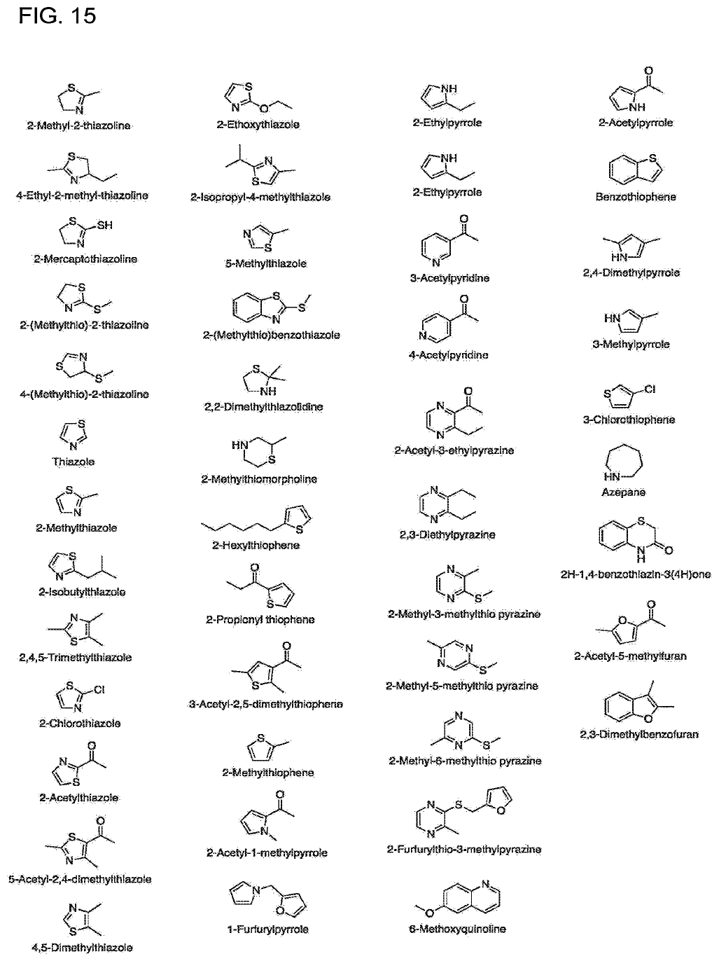

The results are shown in FIG. 14, and the structures of the compounds used are shown in FIG. 15. It was clarified that the administration of the following compounds increases the ratio of neutrophils in the whole blood cells.
4-Ethyl-2-methyl-thiazoline
2-Mercaptothiazoline
2-(Methylthio)-2-thiazoline
2-Methyl-2-thiazoline
4-(Methylthio)-2-thiazoline
Thiazole
2-Methylthiazole
2-Isobutylthiazole
2,4,5-Trimethylthiazole
2-Chlorothiazole
2-Acetylthiazole
5-Acetyl-2,4-dimethylthiazole
4,5-Dimethylthiazole
2-Ethoxythiazole
2-Isopropyl-4-methylthiazole
5-Methylthiazole
2-(Methylthio)benzothiazole
2,2-Dimethylthiazolidine
2-Methylthiomorpholine
2-Hexylthiophene
2-Propionyl thiophene
3-Acetyl-2,5-dimethylthiophene
2-Methylthiophene
2-Acetyl-1-methylpyrrole
1-Furfurylpyrrole
2-Ethylpyrrole
3-Acetylpyridine
4-Acetylpyridine
2-Acetyl-3-ethylpyrazine
2,3-Diethylpyrazine
2-Methyl-(3 or 5 or 6)-methylthio pyrazine (mixture of isomers)
2-Furfurylthio-3-methylpyrazine
6-Methoxyquinoline
2-Acetylpyrrole
Benzothiophene
2,4-Dimethylpyrrole
3-Methylpyrrole
3-Chlorothiophene
Azepane
2H-1,4-benzothiazin-3(4H)one
2-Acetyl-5-methylfuran
2,3-Dimethylbenzofuran
   N≥5, Student's t-test, * P<0.05, ** P<0.01, *** P<0.001

### Example 13

### Experiment Method

About 3-month-old C57/BL6 mice were intraperitoneally injected with 200 μl of saline (control) or 1% solution of respective compounds (dissolved in saline). Blood was collected 16 hr later, and the ratio of neutrophils in the whole blood cells was measured using Siemens Advia 120.

### Results

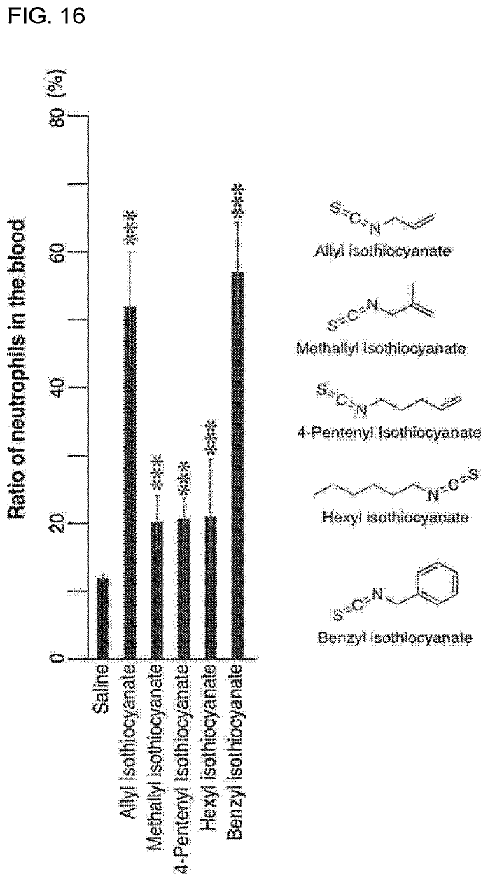

The results and the chemical structures of the compounds used are shown in FIG. 16. It was clarified that the administration of the following compounds increases the ratio of neutrophils in the whole blood cells.
Allyl isothiocyanate
Methallyl isothiocyanate
4-Pentenyl Isothiocyanate
Hexyl isothiocyanate
Benzyl isothiocyanate N≥5, Student's t-test, *** P<0.001

### Example 14

### Experiment Method

About 3-month-old C57/BL6 mice were intraperitoneally injected with 200 μl of saline (control) or 1% solution of respective compounds (dissolved in saline). Blood was collected 16 hr later, and the ratio of basophils in the whole blood cells was measured using Siemens Advia 120.

### Results

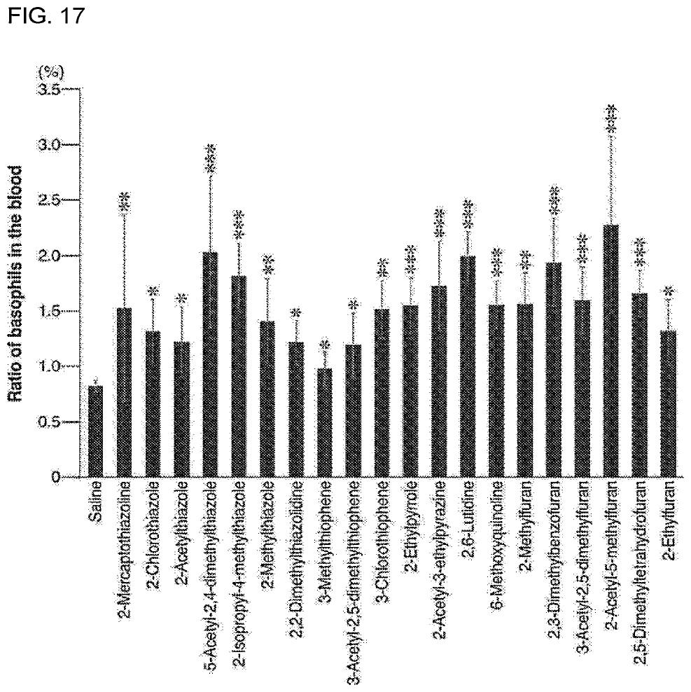

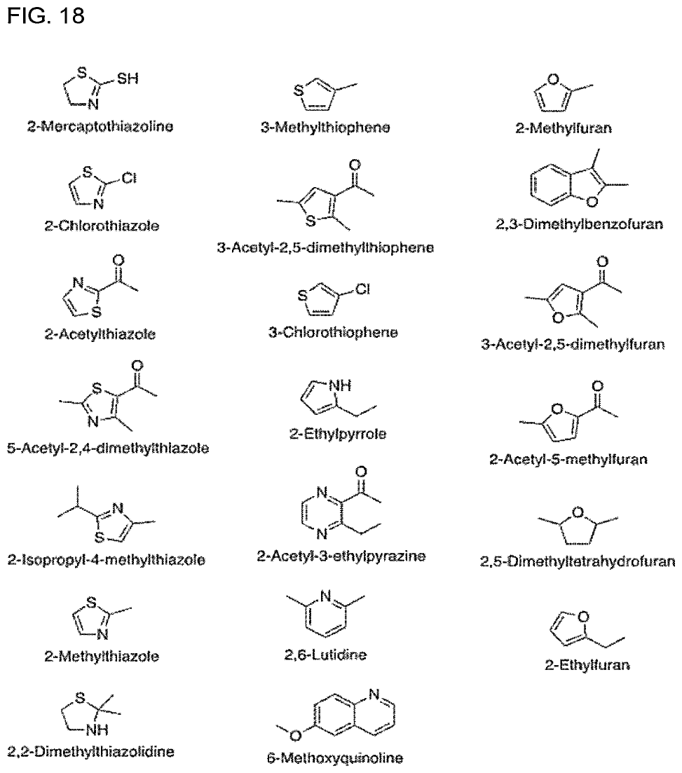

The results are shown in FIG. 17, and the chemical structures of the compounds used are shown in FIG. 18. It was clarified that the administration of the following compounds increases the ratio of basophils in the whole blood cells.
2-Mercaptothiazoline
2-Chlorothiazole
2-Acetylthiazole
5-Acetyl-2,4-dimethylthiazole
2-Isopropyl-4-methylthiazole
2-Methylthiazole
2,2-Dimethylthiazolidine
3-Methylthiophene
3-Acetyl-2,5-dimethylthiophene
3-Chlorothiophene
2-Ethylpyrrole
2-Acetyl-3-ethylpyrazine
2,6-Lutidine
6-Methoxyquinoline
2-Methylfuran
2,3-Dimethylbenzofuran
3-Acetyl-2,5-dimethylfuran
2-Acetyl-5-methylfuran
2,5-Dimethyltetrahydrofuran
2-Ethylfuran
   N≥5, Student's t-test, * P<0.05, ** P<0.01, *** P<0.001

### Example 15

### Experiment Method

About 3-month-old C57/BL6 mice were intraperitoneally injected with 200 of saline (control) or 1% solution of respective compounds (dissolved in saline). Blood was collected 16 hr later, and the ratio of lymphocytes in the whole blood cells was measured using Siemens Advia 120.

### Results

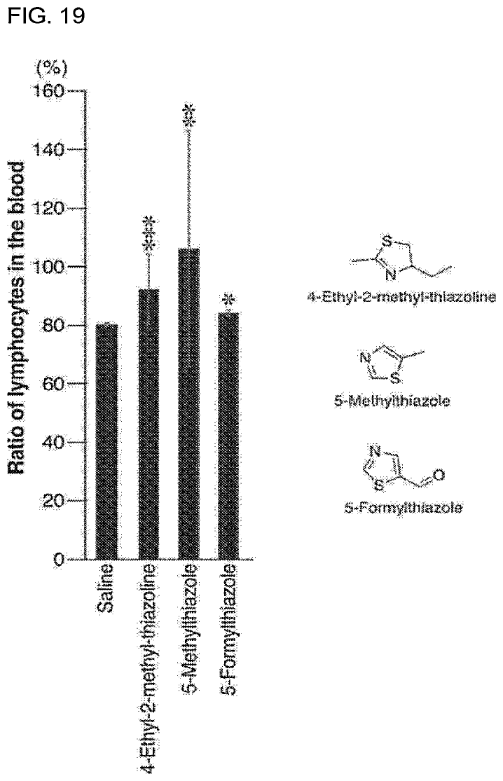

The results and the chemical structures of the compounds used are shown in FIG. 19. It was clarified that the administration of the following compounds increases the ratio of lymphocytes in the whole blood cells.
4-Ethyl-2-methyl-thiazoline
5-Methylthiazole
5-Formylthiazole
   N≥5, Student's t-test, * P<0.05, ** P<0.01, *** P<0.001

### INDUSTRIAL APPLICABILITY

The anti-inflammatory type immune-enhancing agent of the present invention can simultaneously induce an anti-inflammatory action and an immune-enhancing action and can be used to enhance immunity in the prophylaxis or treatment of infections, sepsis, or cancer.

This application is based on patent application No. 2020-054032 filed in Japan, the contents of which are incorporated by reference in full herein.

1.-8. (canceled)

9. A method for enhancing immunity in a mammal, comprising administering, to the mammal, an effective amount of
at least one kind of compound selected from a heterocyclic compound represented by the formula (I) 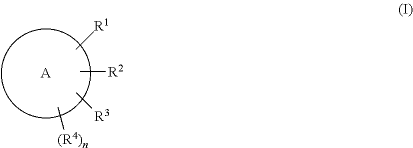
wherein
ring A is a 5- to 7-membered heterocycle containing 1 or 2 hetero atoms selected from a nitrogen atom, an optionally oxidized sulfur atom and an oxygen atom;
R¹, R², R³ and R⁴ are each independently a hydrogen atom, a C₁₋₆ alkyl group, a C₁₋₆ alkoxy group, a halogen atom, an amino group, —SH, a C₁₋₆ alkylthio group, a C₂₋₆ alkenylthio group, a C₁₋₆ alkyl-carbonyl group, a formyl group, a C₆₋₁₀ aryl group, a C₁₋₆ alkoxycarbonyl group, a 5- or 6-membered heteroaryl group, a 5- or 6-membered heteroaryl-C₁₋₆ alkyl group, a 5- or 6-membered heteroaryl-C₁₋₆ alkylthio group, or an oxo group;
R¹ and R² are optionally bonded to each other to form an optionally substituted 5- to 10-membered ring; and
n is 0, 1, or 2, or a salt thereof, and
an isothiocyanate compound represented by the formula (II)
S═C═N—R⁵ (II)
wherein R⁵ is a C₁₋₆ alkyl group, a C₁₋₆ haloalkyl group, a C₂₋₆ alkenyl group, a C₁₋₆ alkylthio-C₁₋₆ alkyl group, a C₆₋₁₀ aryl group, a C₆₋₁₀ aryl-C₁₋₆ alkyl group, or a 5- or 6-membered heteroaryl group.

10. The method according to claim 9 that is a method for enhancing immunity and suppressing inflammation in a mammal.

11. The method according to claim 9, wherein the ring A is thiazoline, thiazole, thiazolidine, thiomorpholine, thiophene, pyrrole, morpholine, azepane, pyridine, pyrazine, furan, 2,3-dihydro-4H-1,4-thiazine, imidazole, or tetrahydrofuran.

12. The method according to claim 9, wherein the compound is a heterocyclic compound represented by the formula (I) or a salt thereof.

13. The method according to claim 9, wherein the compound is an isothiocyanate compound represented by the formula (II).

14. The method according to claim 10, wherein the ring A is thiazoline, thiazole, thiazolidine, thiomorpholine, thiophene, pyrrole, morpholine, azepane, pyridine, pyrazine, furan, 2,3-dihydro-4H-1,4-thiazine, imidazole, or tetrahydrofuran.

15. The method according to claim 10, wherein the compound is a heterocyclic compound represented by the formula (I) or a salt thereof.

16. The method according to claim 10, wherein the compound is an isothiocyanate compound represented by the formula (II).
